(19) United States
(12) Patent Application Publication
(10) Pub. No.: US 2008/0262522 A1
(43) Pub. Date: Oct. 23, 2008

(54) Minimally Invasive Percutaneous Restrictive Bariatric Procedure And Related Device

(76) Inventor: Rachadip Singh Sachasin, Alexandria, LA (US)

Correspondence Address: McGLINCHEY STAFFORD, PLLC, 4703 BLUEBONNET BLVD, BATON ROUGE, LA, 70809, US

(21) Appl. No.: 11/737,887

(22) Filed: Apr. 20, 2007

(51) Int. Cl. A61B 17/122

(52) U.S. Cl. 606/157

(57) ABSTRACT

This invention provides the instrumentation and method to perform a percutaneous restrictive bariatric procedure with very low associated risks as compared to other types of bariatric surgery because the procedure is truly minimally invasive. The procedures of the present invention are performed percutaneously and do not rely on laproscopic or endoscopic guidance. The procedures of the present invention percutaneously restrict the stomach by decreasing the size of the pouch and delay emptying of the pouch by reducing the size of the outlet.

### TECHNICAL FIELD

This invention relates to the medical treatment of obese persons, particularly in the field of percutaneous restrictive bariatric procedures, and related equipment.

### THE INVENTION

Obesity surgery has gained popularity in recent years for the treatment of severely obese people. It is indicated for patients with a body mass index (“BMI”) of 40 and greater or 35 and greater if co-morbidities, such as diabetes, heart disease and hypertension are present. BMI is obtained by dividing a person's weight in kilograms by height in meters square.

Patients with BMI greater than 40 are considered morbidly obese and are greater than 100 pounds overweight for men and greater than 80 pounds overweight for women. Patients with BMI of 35-40 are considered severely obese. Both of these groups of patients are considered to have very high or extremely high levels of health risks. With known surgical devices and methods, bariatric surgery is performed on the morbidly obese; however, the presence of comorbities, or medical illnesses or diseases that are either caused by or contributed to by morbid obesity such as diabetes, high blood pressure, high cholesterol, sleep apnea, and arthritis, lowers the weight threshold for known surgical treatment to include the severely obese. To date, most bariatric surgery is usually undertaken in only these two groups, the morbidly obese and the severely obese with comorbities, since the benefits of bariatric surgery outweigh the risks in these particular patients.

There is, however, a large segment of the population that would potentially benefit from bariatric surgery. This is the segment with a BMI of 30-35 or BMI of 35-40 with no or minimal comorbidities. Approximately 25% of the adult American population is obese with a BMI of greater than 30. The segment of the population with BMI of 30-35 and BMI of 35-40 with a low number for comorbidities accounts for the majority (80% of total) of obese Americans. Associated health risks for this population is not as high as the morbidly obese, however, their risk is still classified as high. For people with a BMI of greater than or equal to 30, all cause mortality, increased by 50%-100% above that of people with BMI in the range of 20-25. Due to the associated risk of bariatric surgery, this large segment of the population has failed to qualify for surgical weight loss therapy. The risks simply outweigh the benefits in this population.

Bariatric surgery alters the digestive process and is classified into two categories: restrictive and malabsorbitive procedures. Each procedure is discussed in turn below.

Restrictive procedures promote weight loss by closing off parts of the stomach to make it smaller, thus restricting the amount of food the stomach can hold. These procedures do not interfere with the normal digestive process. The surgery essentially creates a small pouch at the top of the stomach where food enters from the esophagus. The outlet of the pouch usually has a diameter of approximately three-quarter to one inch and restricts/delays the emptying of food from the pouch and causes a feeling of fullness. As a result of this surgery, most patients lose the ability to eat large amounts of food at one time. Restrictive operations include adjustable gastric banding and vertical banded gastroplasty. In the adjustable gastric banding procedure, a hollow band is placed around the stomach near the upper end, creating a small pouch and a narrow passage into the remainder of the stomach. The band is inflated with saline and can be tightened or loosened over time to change the size of the outlet. The vertical banded gastroplasty has been the most common restrictive operation for weight control. In this procedure, both a band and staples are used to create a small stomach pouch. Restrictive procedures are frequently performed laparoscopically and are associated with a lower number of risks and complications than are the malabsorbative procedures.

Malabsorptive procedures were traditionally the most common surgeries for weight loss, but have become less popular recently, as the risk and complication rate for these procedures is significantly higher than restrictive surgery. These procedures combine stomach restriction with a partial bypass of the small intestine. A direct connection from the stomach to the mid or lower segment of the small intestine is created, bypassing portions of the digestive tract that absorb calories and nutrients. These procedures include the Roux-en-Y gastric bypass and the biliopancreatic diversion. Although malabsorbative procedures successfully promote weight loss, they are highly invasive, can also lead to nutritional deficiencies, and are associated with increased morbidity and mortality. For this reason, the popularity of malabsorbitive procedures has decreased recently, especially since restrictive surgeries can be performed laparoscopically and are associated with significantly decreased risks and complications. Additionally, these procedures generally are not reversible.

Risks of bariatric surgery include pulmonary embolism from clots in the legs, staple line breakdowns and leaks, wound infections or seromas (fluid collections in the soft tissues of the abdominal wall). The outlet that is created from the surgery can also become inflamed and narrowed, usually from vomiting following the procedure. The gastric band may become dislodged or slip. Complications resulting from general anesthesia, especially intubation are particularly common in patients greater than 400 pounds.

Approximately 20% of patients require admission to the Intensive Care Unit (“I.C.U.”) following a bariatric procedure. Obesity is a risk factor for pulmonary embolism and pulmonary embolism is the most common postoperative complication. This is exacerbated by the fact that the patients undergoing bariatric surgery are often not able to ambulate quickly after the surgery. This leads to an increased incidence of clot and thrombus forming in the legs, which eventually becomes dislodged and embolizes to the lungs. Incisional hernias are also known complications of traditional open bariatric procedures.

Morbidity and mortality have been reduced in recent years because of more surgeries being performed laparoscopically. Laparoscopic procedures are considered minimally invasive. Although laparoscopic procedures are considered minimally invasive, they still require general anesthesia and some surgical incisions and relatively large instruments when compared to other minimally invasive procedures. Because of these limitations, laparoscopic procedures are still reserved for patients with a BMI greater than 40 or greater than 35 with associated comorbidities.

One example of a restrictive, laparoscopic bariatric procedure is US 2006/0212053 to Gertner. However, Gertner uses different methods and devices of the present invention and is more invasive than the present invention.

Unlike known bariatric surgical procedures, the present invention provides methods and devices for performing percutaneous bariatric surgery that is truly minimally invasive and that is relatively easily reversible. Percutaneous interventions are performed via small incisions (generally 1-2 cm) into the skin and usually rely on imaging guidance, such as fluoroscopy (continuously moving X-rays), ultrasound or CT scans to guide the physician during the procedure. These procedures do not require general anesthesia and are very well-tolerated by patients. Morbidity and mortality for these percutaneous minimally invasive procedures is significantly less than traditional surgery and laparoscopic procedures. They are less expensive than surgical procedures and are usually performed on an outpatient basis. Rarely an overnight hospital stay is required for observation. The present invention includes the instrumentation and method to perform a percutaneous restrictive bariatric procedure and is truly minimally invasive because it does not rely on laproscopic or endoscopic guidance. Also, the present invention is relatively easily reversible, unlike most known bariatric surgical procedures.

More particularly, this invention provides the instrumentation and method to perform a percutaneous restrictive bariatric procedure. The purpose of the procedure is to percutaneously restrict the stomach by decreasing the size of the pouch and delay emptying of the pouch by reducing the size of the outlet. Associated risks of the procedure are very low when compared to other types of bariatric surgery because the procedure is truly minimally invasive because it is performed percutaneously and because it does not rely on laproscopic or endoscopic guidance. Because of a decreased morbidity associated with the present invention, physicians will be able to perform the procedure of the present invention on the largest segment of the obese population patients with BMI of 30-40, since the benefits will outweigh the risks associated with the procedure. Also, due to the increased safety profile of this procedure, it can be considered in obese children.

The purpose of the procedure is to percutaneously restrict the stomach by decreasing the size of the pouch and delay emptying of the pouch by reducing the size of the outlet.

Thus, one embodiment of this invention is a method of performing a bariatric procedure comprising piercing a wall of a stomach, the stomach defining a passageway; disposing at least a portion of a restricting device through the pierced wall, actuating the restricting device to draw the pierced wall and an opposing wall together in a passageway-restricting position, with or without piercing the opposing wall; anchoring the restricting device to the stomach to maintain the pierced and opposing stomach walls in the passageway-restricting position.

Another embodiment of the present invention is a method of performing a bariatric procedure further comprising inhibiting contact between an inner surface of the pierced stomach wall and an inner surface of the opposing stomach wall while the pierced and opposing stomach walls are drawn together and anchored in a passageway-restricting position.

Still another embodiment of the present invention is a method of performing a bariatric procedure further comprising inserting a spacer between the pierced stomach wall and the opposing stomach wall to maintain a prescribed distance between the inner surface of the pierced stomach wall and the inner surface of the opposing wall while in a passageway-restricting position.

Another embodiment of the present invention is the method of performing a bariatric procedure as described above wherein the step of anchoring the restricting device to the stomach comprises attaching at least one wall restraining anchor to the opposing stomach wall.

Yet another embodiment of the present invention is a method of performing a bariatric procedure further comprising advancing a retention device against an outside surface of the pierced wall of the stomach until the inner surface of the pierced wall of the stomach abuts at least a portion of a surface of the spacer, thereby bringing the pierced wall and the opposing wall of the stomach closer together in the passage-restricting position, while inhibiting adhesion between mucosa on the pierced and opposing walls of the stomach.

Another embodiment of this invention is a medical device comprising at least one anchor, an anchor deployment device, at least one filament, a spacer having a three-dimensional form and comprising a top portion and a bottom portion, and a retention strip forming ridges on at least one surface of the retention strip, wherein the anchor is connected to the filament and the filament is connected to the retention strip, and wherein the anchor deployment device is sized and configured to be at least partially threaded over the anchor, the filament, and the retention strip so that, during use, the anchor can be deployed from the anchor deployment device into a stomach wall to a deployed position wherein the filament is attached to the anchor, the retention strip is attached to the filament, and the anchor deployment device is removable from a position surrounding the anchor, the attached filament, and the retention strip while the anchor, the filament, and the retention strip remain in the deployed position.

Yet another embodiment of the present invention is the medical device as described above wherein the filament is comprised of a plastic material and has a diameter of approximately a 0-0 suture.

Still another embodiment of the present invention is the medical device as described above wherein the retention strip is comprised of a plastic material and wherein the ridges on the surface of the retention strip are sized and configured to operatively connect to a retention head in at least one opening of a retention device in order to allow irreversible ratcheting movement of the retention device down the retention strip.

Another embodiment of the present invention is the medical device described above wherein the anchor is comprised of a plastic material and forms one or more gripping teeth on at least one anchor surface.

Yet another embodiment of the present invention is the medical device described above further comprising a spacer having a three-dimensional form and comprising a top portion and a bottom portion, where in the spacer defines at least one slit opening extending through the bottom portion and at least one slit opening extending through the top portion, each slit opening being sized and configured so that at least one retention strip and the filament can be threaded through the spacer.

Still another embodiment of the present invention is the medical device described above wherein the spacer further defines a window on each of two longitudinal sides, wherein the windows are sized and configured to provide access to the filament when the filament is disposed within the spacer and it is desired to sever the filament with a cutting device.

Another embodiment of the present invention is a medical device comprising a rigid backing and a collagen plug connected thereto.

Yet another embodiment of the present invention is the medical device described above wherein the rigid backing and the collagen plug each define a respective opening, wherein the opening defined by the rigid backing is substantially aligned with the opening defined by the collagen plug.

Still another embodiment of the present invention is the medical device described above wherein the opening defined by the rigid backing contains at least one retention head, wherein said retention head is sized and configured to catch at least one ridge on a surface of a retention strip when the retention strip is threaded through the opening defined by the rigid backing into a retention position, to thereby retain the retention strip in place.

These and other embodiments and features of this invention will be still further apparent from the ensuing description, the accompanying drawing figures and appended claims.

### BRIEF DESCRIPTION OF THE DRAWINGS

FIGS. 1A-1O illustrate one embodiment of a method of the percutaneous bariatric surgical procedure of the present invention.

FIG. 2 illustrates one embodiment of a medical device of the present invention comprising an anchor, a filament, and a retention strip.

FIG. 3 illustrates one embodiment of a medical device of the present invention comprising an outer tube, a plunger, anchors, filaments, and retention strips.

FIG. 4 illustrates one embodiment of a medical device of the present invention comprising a rigid backing, a collagen plug, and two retention heads.

Like reference numbers or letters are employed within the various figures to refer to like parts or components.

### FURTHER DETAILED DESCRIPTION OF THE INVENTION

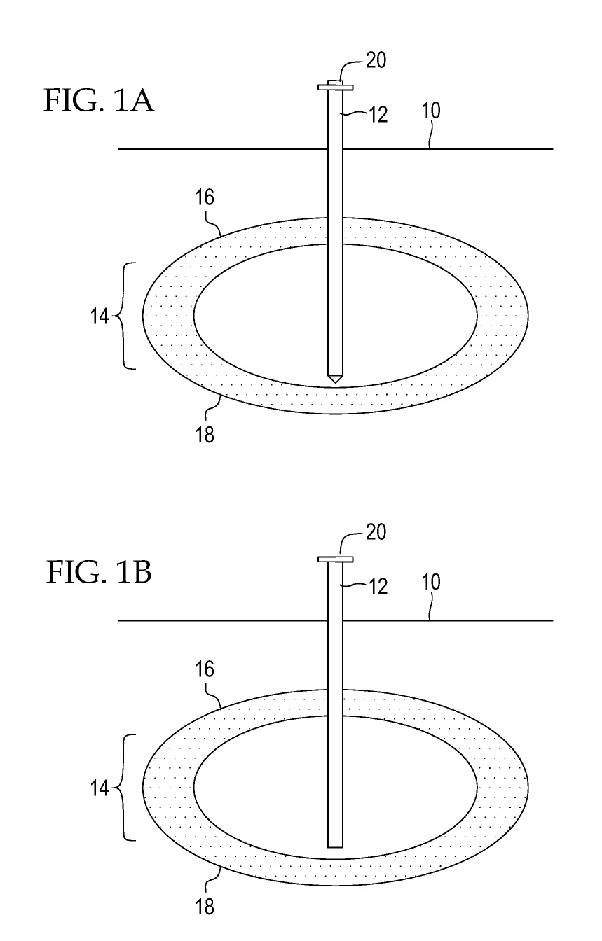

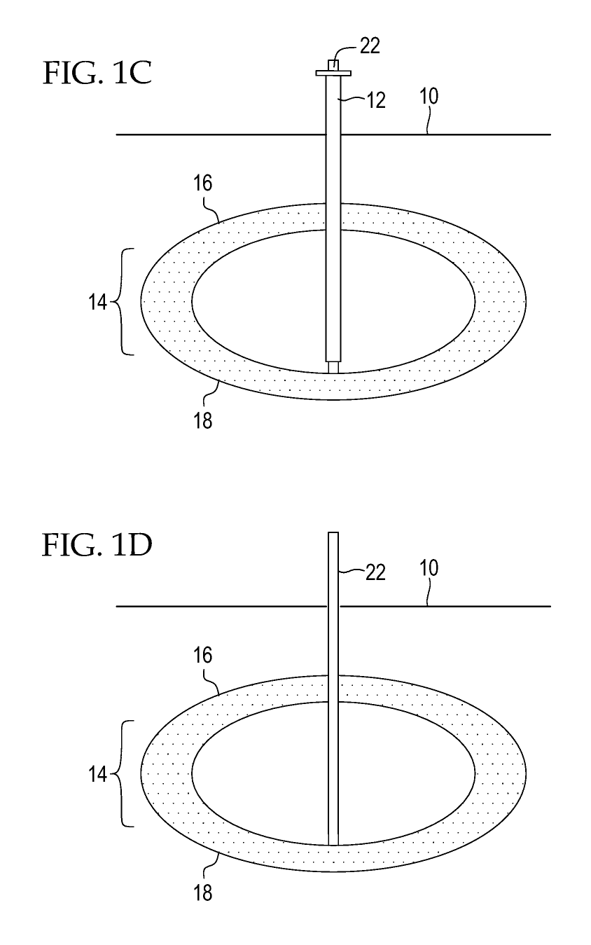

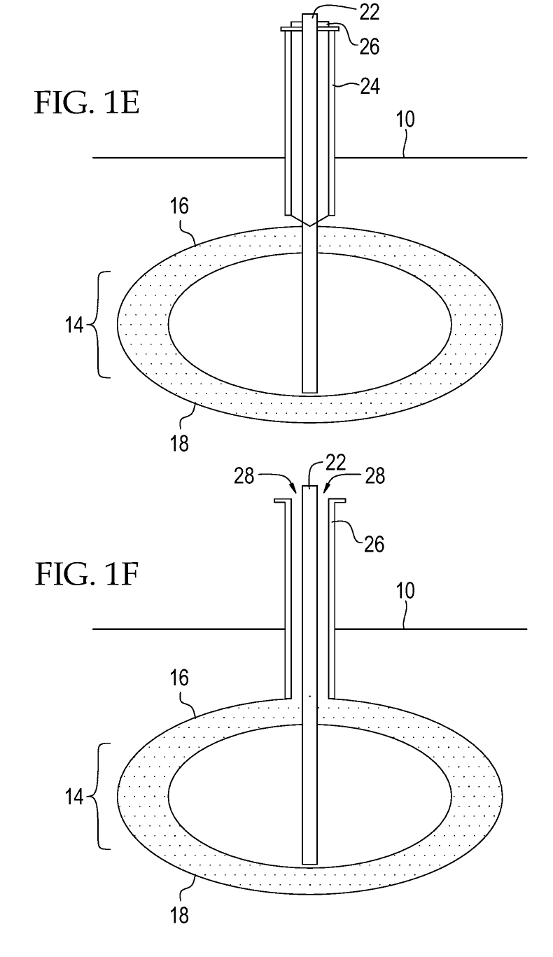

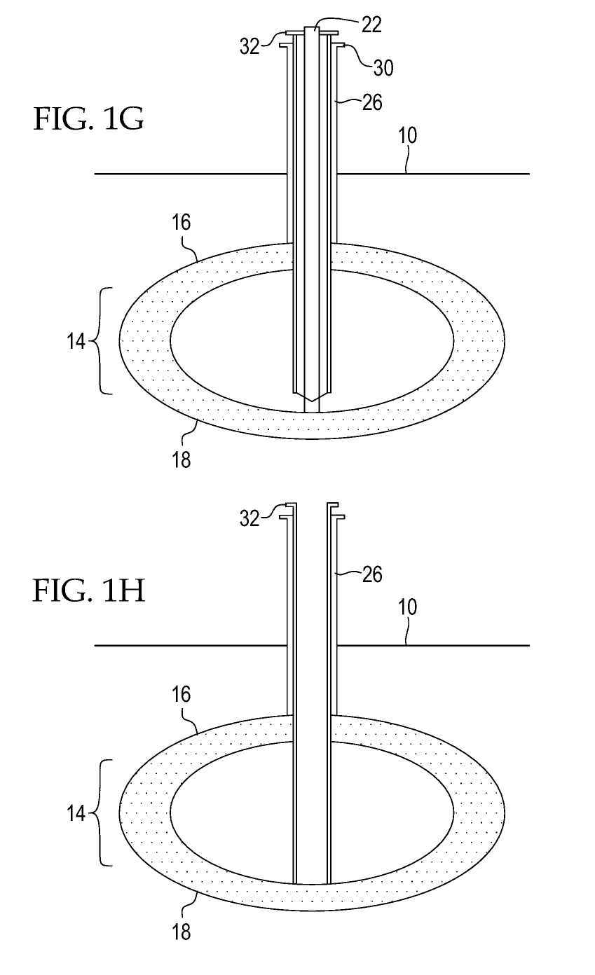

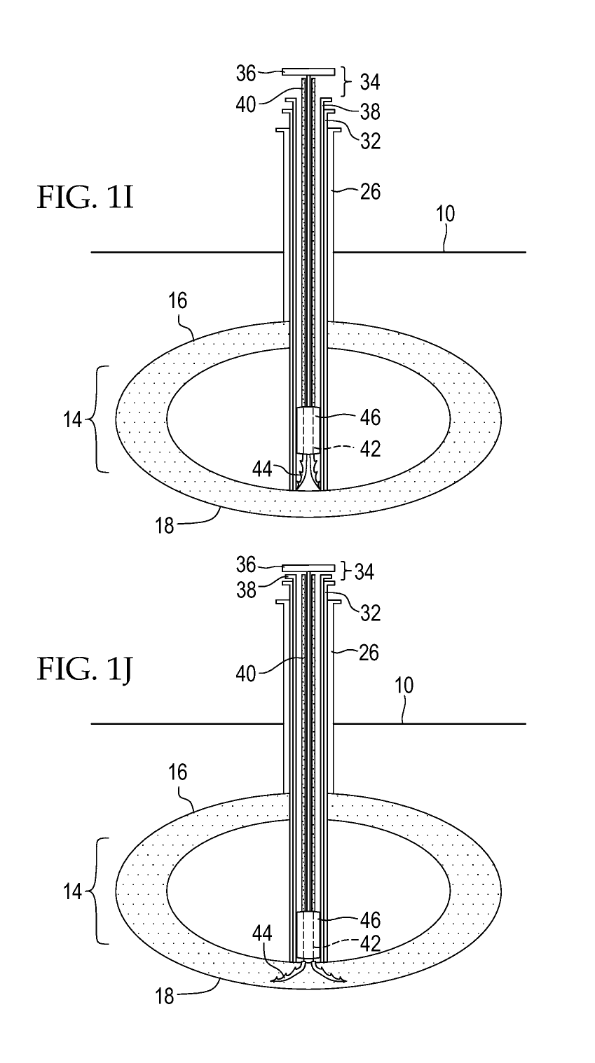

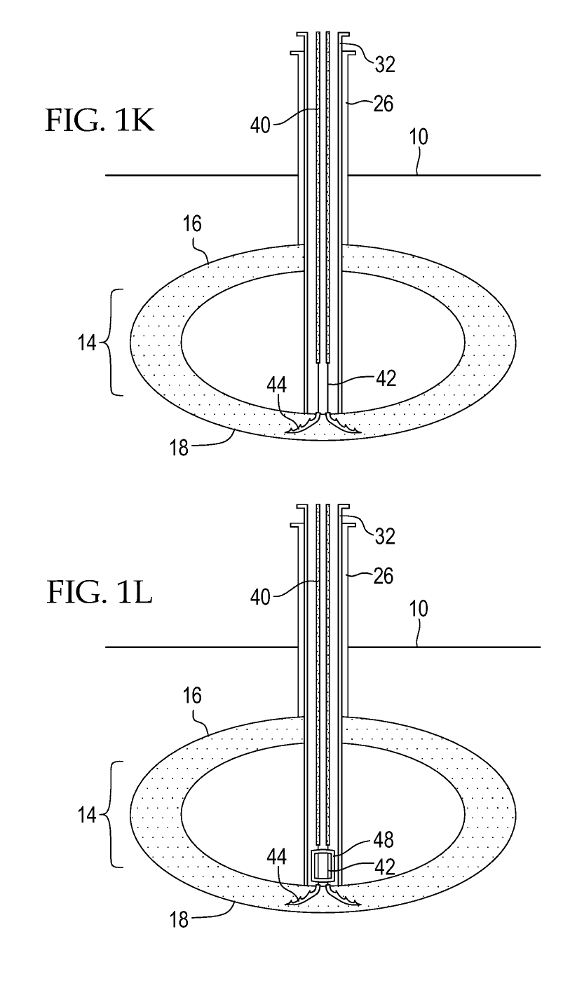

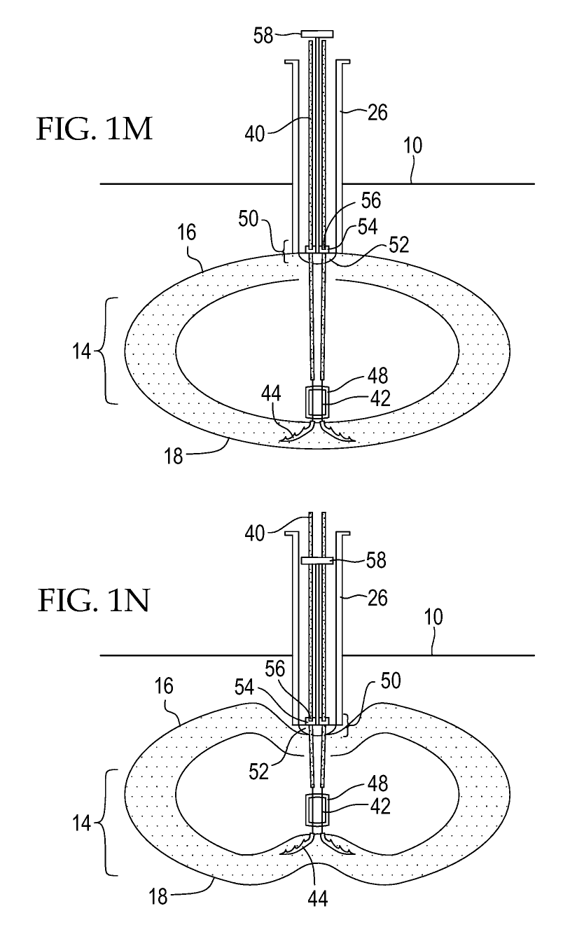

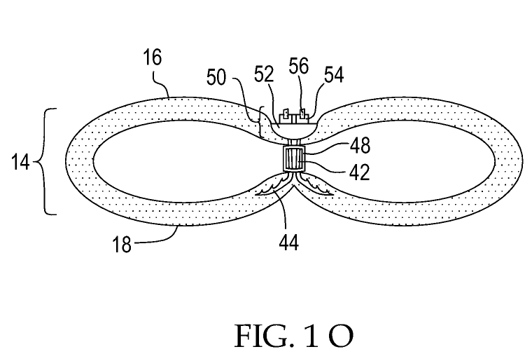

FIGS. 1A-1O illustrate one embodiment of a method of the percutaneous bariatric surgical procedure of the present invention.

In preparation for the method described below and as seen in FIG. 1A-1O, a nasogastric tube is advanced into the stomach, and the stomach is insufflated with air. A small amount of water soluble radiographic contrast can be introduced into the stomach, if needed, for better visualization of the gastric walls.

Then, as can be seen in FIG. 1A, one embodiment of performing the percutaneous bariatric procedure of the present invention comprises making an incision in the skin overlying the anterior abdominal wall 10, advancing percutaneously, under fluoroscopy, an access needle with outer sheath 12 and a removable inner stylet 20 at least through the anterior abdominal wall 16 and through an anterior or pierced stomach wall 16, with or without piercing a posterior or opposing stomach wall 18, thereby creating a hole in the anterior stomach wall 16.

Next, the inner stylet 20 of the access needle with outer sheath 12 is removed, as can be seen in FIG. 1B, leaving only the access needle with outer sheath 12. As can be seen in FIG. 1C, the inner stylet 20 of the access needle 12 is replaced with a sufficiently blunt-ended guiding pin 22. Then, the outer sheath of the access needle 12 is removed, leaving only the guiding pin 22 in place, as can be seen in FIG. 1D, which guiding pin 22 does not pierce the posterior stomach wall 18.

As illustrated in FIG. 1E, at least one first dilator 24 is positioned over the guiding pin 22, dilating sufficiently serially the anterior abdominal wall 10 with the first dilator 24, advancing a large introducer trochar 26 with an inner dilator over the guiding pin 22 to the anterior stomach wall 16. Next, the inner dilator is removed from around the large introducer trochar 26, allowing the large introducer trochar 26 to rest on the anterior stomach wall 16, as can be seen in FIG. 1F, which allows for an access portal 28 for the remainder of the procedure.

Next, as illustrated in FIG. 1G, a second dilator 30 over the guiding pin 22, wherein the anterior stomach wall 16 incision is dilated sufficiently to accommodate a small introducer trochar 32, and wherein the small introducer trochar 32 with an inner dilator is advanced over the guiding pin 22 through the anterior stomach wall 16, allowing the small introducer trochar 32 to come to rest at the posterior wall 18 of the stomach 14.

As illustrated in FIG. 1H, the guiding pin 22 and the inner dilator of the small introducer trochar 32 are removed, and the small introducer trochar 32 is pushed against the posterior stomach wall 18 inside the stomach 14. Then, as shown in FIG. 1I, a preloaded anchor deployment system 34 is introduced into the stomach through the small introducer trochar 32, wherein the preloaded anchor deployment system 34 is comprised of at least two anchors 44, each with attached filaments 42 (which are hidden in FIG. 1I under an anchor deployment device 46) and retention strips 40, an anchor deployment device 46, a deployment plunger 36, and an outer tube 38 encasing at least part of the preloaded anchor deployment system 34.

Then, as illustrated in FIG. 1J, the preloaded anchor deployment system 34 is deployed by pushing the preloaded anchor deployment system 34 firmly against the posterior stomach wall 18, depressing the deployment plunger 36 on the preloaded anchor deployment system 34, which deploys the anchors 44 with attached filaments 42 and retention strips 40, which are threaded through the plunger 36, into the posterior stomach wall 18.

Next, as illustrated in FIG. 1K, the plunger 36 (from FIG. 1J) is removed, leaving behind the filaments 42 and retention strips 40 attached to the anchors 44. Then, as illustrated in FIG. 1L, a spacer 48 is threaded with the retention strips 40, the spacer 48 is pushed down the retention strips 40 and the filaments 42 until the spacer 48 touches the posterior stomach wall 18. Then, the small introducer trochar 32 is removed.

As illustrated in FIG. 1M, a plug retention device 50 is threaded onto the retention strips 40. The plug retention device 50 is comprised of a collagen plug 52 attached to a rigid backing 54 with retention heads 56. Using a pusher device 58, which is comprised of a plunger, the plug retention device 50 is pushed through the large introducer trochar 26 to the anterior stomach wall 16. Depressing the plunger of the pusher device 58 while holding the retention strips 40 stationary effectively pulls the anterior and posterior walls of the stomach together as the pusher device 58 tightens by irreversibly ratcheting on the retention heads 56 which are sized and configured to catch on at least one ridge on the surface of the retention strips 40. As the retention strips 40 are tightened, the collagen plug 52 is seated over the hole in the anterior stomach wall 16. Then, as illustrated in FIG. 1N, as the anterior stomach wall 16 and the posterior stomach wall 18 are drawn together. When the anterior stomach wall 16 and the posterior stomach wall 18 have been sufficiently drawn together, the pusher device 58 (from FIG. 1M) is removed.

As can be seen in FIG. 1O, contact between an inner surface of the anterior stomach wall 16 and the posterior stomach wall 18 is inhibited with the spacer 48, which will prevent fusing and/or scarring between the anterior stomach wall 16 and the posterior stomach wall 18 and will allow for reversal of the procedure.

As illustrated in FIG. 1N, after the plug retention device 50 is sufficiently tightened over the retention strips 40, the retention strips 40 are cut and removed. Then, the large introducer trochar 26 is removed, and the incision in the skin in the anterior abdominal wall 10 is closed with sutures.

Then, the preceding steps shown in FIGS. 1A-1N are repeated, e.g., three to five times, along the lesser curvature of the stomach and possibly along the left costal margin as it overlies the fundus of the stomach. The procedure of this embodiment of the present invention, as it relates to the lesser curvature, is performed along the body of the stomach, but not the antrum. This results in a small narrow channel from the gastroesophageal junction to the antrum. Food enters the stomach and moves down the path of least resistance. This path is along the lesser curvature of the stomach since the fundus and most of the body have effectively been excluded. Food goes into the smaller narrow lumen resulting in early satiety and emptying is delayed because the outlet has been reduced to small holes, typically (but not necessarily) approximately one inch in size. The pouch is smaller and the outlet has essentially become a sieve. Digestion and the production of chime is not affected since the antrum is left intact. This will result in weight loss much in the same way other restrictive bariatric procedures do. However, it is reversible and will not result in the kind of scarring and adhesion of the stomach walls to each other experienced in other restrictive procedures.

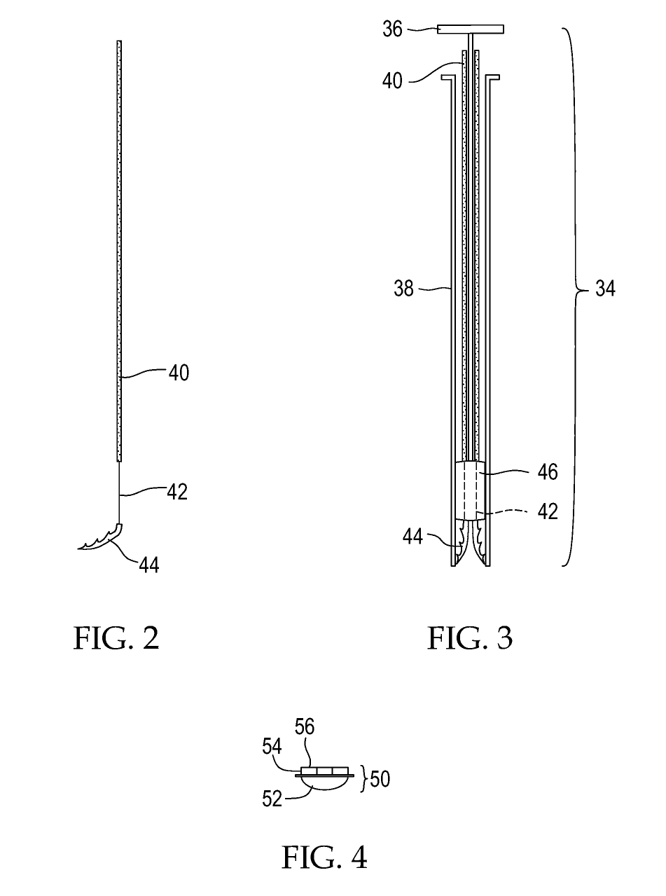

FIG. 2 illustrates one embodiment of a medical device of the present invention comprising an anchor 44, a filament 42, and a retention strip 40, wherein the anchor 44 is operatively connected to the filament 42 which is operatively connected to the retention strip 40.

FIG. 3 illustrates one embodiment of a medical device of the present invention, namely a preloaded anchor deployment system 34, comprising two anchors 44, each with attached filaments 42 (which are hidden in FIG. 3 under an anchor deployment device 46) and retention strips 40, an anchor deployment device 46, a deployment plunger 36, and an outer tube 38 encasing at least part of the preloaded anchor deployment system 34.

FIG. 4 illustrates one embodiment of a medical device of the present invention, namely a plug retention device 50, comprising a collagen plug 52 attached to a rigid backing 54 with retention heads 56.

As discussed supra, in one embodiment of the present invention, a method of performing a bariatric procedure comprises piercing a wall of a stomach. The piercing of a wall of the stomach may be accomplished in various ways in accordance with the present invention, including using an access needle with outer sheath and a removable inner stylet itself to pierce the stomach wall, initially using an introducer device to puncture or pierce the tissue of the stomach wall, or like procedures. The preferred method of piercing the stomach wall is using an access needle itself with outer sheath and a removable inner stylet. Any suitable access needle may be used in accordance with the present invention. The access needle used may be chosen from such specialty and access needles as a needle with inner stylet, a sheathed needle, or simple introducer or access needle, and the like, wherein the specialty and access needles may be of various gauges and comprised of various materials. Most preferred is a straight, 11-gauge, stainless steel access needle with a beveled or cutting edge on one end of the access needle, the length of which access needle is selected depending on the size of the patient. In a preferred embodiment of the present invention, when the inner stylet of the access needle is removed, the outer sheath of the access needle will be of sufficient inner diameter to allow a guiding pin to be inserted in the inner stylet's place. The guiding pin of the present invention is preferably a solid pin with blunt ends, comprised of stainless steel, and the guiding pin is preferably has same diameter as the inner stylet of the access needled and can be various lengths depending on the size patient, but approximately is 30 cm in length.

Also in this embodiment of the present invention, the step of disposing at least a portion of a restricting device through the pierced wall may be accomplished first by using any medical device which creates a sufficiently-sized access portal through the anterior abdominal wall and the pierced stomach wall. Preferably, at least one dilator is positioned over the guiding pin and dilated, thereby dilating the anterior abdominal wall sufficiently to advance a large introducer trochar with inner dilator over the guiding pin. This step may be accomplished by advancing multiple dilators of increasing-diameter through the anterior abdominal wall and then dilating each over the guiding pin until the large introducer trochar with inner dilator may be advanced. Most preferably, an initial dilator only slightly larger than the guiding pin will be used, followed by successively larger dilators, serially dilating the anterior abdominal wall until the wall is sufficiently dilated to accommodate a large introducer trochar with inner dilator, wherein the inner dilator has an inner diameter that is of a sufficient size to accommodate the guiding pin through its center and wherein when the inner dilator is removed, the large introducer trochar has an internal diameter that is of sufficient size to accommodate a smaller trochar and a restricting device as well as any deployment device for the restricting device through its interior. Most preferably, the large introducer trochar will be a size 24 French trochar. Also, while the dilators and inner dilator of the large introducer trochar may be comprised of one or more of a wide variety of materials, preferably, dilators and inner dilators used to dilate the anterior abdominal wall are comprised of a plastic or a metal material suitable for use in surgical procedures. More preferably, the dilators used to dilate the anterior abdominal wall are comprised of plastic suitable for use in surgical procedures, and the inner dilator of the large introducer trochar is more preferably a metal material suitable for use in surgical procedures and most preferably stainless steel.

Then, preferably, the same procedure for serially dilating the pierced stomach wall is performed by dilating the incision in the pierced stomach wall by starting with a dilator only slightly larger than the guiding pin and then successively larger dilators, serially dilating the pierced stomach wall until the wall is sufficiently dilated to accommodate a small introducer trochar with inner dilator over the guiding pin thereby creating an access portal into the stomach, wherein the inner dilator has an inner diameter that is of a sufficient size to accommodate the guiding pin through its center and wherein when the inner dilator is removed, the small introducer trochar has an internal diameter that is of sufficient size to accommodate the restricting device as well as any deployment device for the restricting device through its interior. Most preferably, the small introducer trochar will be a size 20 French trochar. Also, while the dilators and inner dilator of the small introducer trochar may be comprised of one or more of a wide variety of materials, preferably, dilators and inner dilator used to dilate the pierced stomach wall are comprised of a plastic or a metal material suitable for use in surgical procedures. More preferably, the dilators used to dilate the pierced stomach wall are comprised of plastic suitable for use in surgical procedures, and the inner dilator of the small introducer trochar is more preferably a metal material suitable for use in surgical procedures and most preferably stainless steel.

Preferably, a preloaded anchor deployment system of this invention or similar device is used to dispose the restricting device through the anterior abdominal wall and then the pierced stomach wall through the interior of the trochar. The preloaded anchor deployment system preferably is comprised of a restricting device and a device for deploying the restricting device. The restricting device of this invention can be any device that can restrict the walls of the stomach to form a passageway. Preferably, the restricting device comprises at least one anchor, at least retention device, at least one filament, and at least one retention strip with ridges on at least one surface of the retention strip, wherein the anchor is connected to the filament and the filament is connected to the retention strip. The device for deploying the restricting device is preferably a pusher device and an anchor deployment device. Preferably, the anchor deployment device is sized and configured to be at least partially threaded over the anchor, the filament, and the retention strip so that, during use, the anchor can be deployed from the anchor deployment device into a stomach wall to a deployed position wherein the filament is attached to the anchor, the retention strip is attached to the filament, and the anchor deployment device is removable from a position surrounding the anchor, the attached filament, and the retention strip while the anchor, the filament, and the retention strip remain in the deployed position. Preferably, the filament of this invention is comprised of a plastic material suitable for use in surgical procedures or non-absorbable suture material such as silk and has a diameter of approximately a 0-0 suture. Preferably, the retention strip is plastic and is approximately 2 mm thick.

The step of actuating the restricting device to draw the pierced wall and an opposing wall together in a passageway-restricting position, with or without piercing an exterior surface of the opposing wall, is performed by using at least one anchor and/or retention device in two opposing walls of the stomach, the pierced wall and the opposing wall, whereby the anchor and/or retention device are connected in some manner which allows the walls of the opposing walls of the stomach to be drawn together. Also, while the exterior surface of the opposing wall may or may not be pierced and still be within the scope of this invention, it is preferred that the exterior surface of the opposing wall not be pierced while performing this method. While the pierced and opposing walls of the present invention may be any two opposing walls of the stomach, in the preferred embodiment the pierced wall is the anterior stomach wall, while the opposing wall is the posterior stomach wall. Preferably, at least one retention strip and connected filament operatively connect the anchor and/or retention devices in the two opposing walls of the stomach. Most preferably, two retention strips and connected filaments connect a retention device in the pierced wall to an anchor in the opposing wall. In this most preferred embodiment, the retention device may be any medical device which allows the pierced and opposing stomach walls to be drawn together in a passageway-restricting position. Preferably, the retention device comprises a rigid backing and a plug connected thereto, and preferably, the rigid backing and the plug each define at least one respective opening, wherein the opening defined by the rigid backing is substantially aligned with the opening defined by the plug. In a preferred embodiment, the opening defined by the rigid backing contains at least one retention head, wherein said retention head is sized and configured to catch at least one ridge on a surface of a retention strip when the retention strip is threaded through the opening defined by the rigid backing into a retention position, to thereby retain the retention strip in place. In the most preferred embodiment, the retention device has two retention heads, each of which is similarly sized and configured to catch at least one ridge on a surface of a retention strip when the retention strip is threaded through each opening defined by the rigid backing into the passageway-restricting position. Preferably, the retention strips are comprised of a plastic material with ridges on at least one surface of the retention strips. Most preferably, a retention device with plug is threaded with the retention strips, and as the retention strips are stabilized, preferably by holding one in each hand, a pusher device comprised of a plunger is used by making contact with a flat surface of the rigid backing, preferably between two retention heads, and pushing the retention device with plug against an exterior surface of the pierced stomach wall, and as the plunger is depressed, the retention device with plug is irreversibly ratcheted down the retention strips effectively drawing the pierced and opposing stomach walls together into a passageway-restricting position. The passageway-restricting position can be any position in which the passageway of the stomach is reduced, preferably by drawing two opposing stomach walls together to form a restricted passageway, and more preferably, the two opposing stomach walls are brought together, but not allowed to touch when in the passageway-restricting position. Preferably, the plunger is comprised of plastic or steel.

In this embodiment of the present invention, the step of anchoring the restricting device to the stomach to maintain the pierced and opposing stomach walls in the passageway-restricting position can be accomplished in various ways. Preferably, the step of anchoring utilizes at least one wall restraining anchor as the anchoring device in the opposing stomach wall, while a plug retention device is used with the pierced stomach wall to push the pierced stomach wall while the anchor pulls the opposing stomach wall into a passageway-restricting position. Preferably, the plug portion of the retention device is slightly larger than the hole made in the pierced stomach wall, so the plug will be seated over the hole to effectively seal the hole and prevent any leakage. Any device which can anchor into the opposing wall of the stomach may be used, but most preferably, an exterior surface of the opposing stomach wall may or may not be pierced when the anchor is deployed in the opposing wall. The anchor and retention device can be made of any biocompatible material. Most preferably, the plug portion of the retention device is made of a collagen material, as a collagen plug would also help seal the stomach during the healing process. Preferably, more than one anchor is deployed to attach the restricting device to the opposing stomach wall. Most preferably, two anchors are deployed into the opposing stomach wall. The anchors may be comprised of any biocompatible material and any means to attach to the opposing stomach wall with or without piercing an outer surface of the opposing outer wall. Preferably, the anchors are intrinsically curved, are comprised of plastic material, and form one or more gripping teeth on at least one surface of the anchor to secure the anchors in the opposing stomach wall with or without piercing an outer surface of the opposing stomach wall. Maintaining the pierced and opposing stomach walls in the passageway-restricting position can be accomplished in various ways. Preferably, the retention device is irreversibly ratcheted down the retention strips toward the opposing wall using ridges on the surface of the retention strip operatively connected to a retention head in at least one opening of the retention device, so that when the prescribed distance between the opposing stomach walls is achieved, ratcheting can be stopped and the irreversible nature of the retention strips will allow the prescribed distance between the opposing stomach walls to be maintained.

In a preferred embodiment of the present invention, the step of inhibiting contact between an inner surface of the pierced stomach wall and an inner surface of the opposing stomach wall while the pierced and opposing stomach walls are drawn together and anchored in a passageway-restricting may be accomplished in a number of ways. Preferably the walls are inhibited from touching by maintaining a sufficient distance between the two walls by controlling the length of retaining strip which is threaded through the opening in the retention device with plug or by using a spacer as of this invention between the two opposing stomach walls. The most preferred method of inhibiting contact is using a spacer. The spacer is inserted between the pierced stomach wall and the opposing stomach wall to maintain a prescribed distance between the inner surface of the pierced stomach wall and the inner surface of the opposing wall while in a passageway-restricting position. Preferably, the plug retention device is advanced against an outside surface of the pierced wall of the stomach until the inner surface of the pierced wall of the stomach abuts at least a portion of a surface of the spacer, thereby bringing the pierced wall and the opposing wall of the stomach closer together in the passage-restricting position, while inhibiting adhesion between mucosa on the pierced and opposing walls of the stomach. The spacer can be any three-dimensional form and of a sufficient size that is able to inhibit contact between the opposing walls of the stomach when the walls and drawn together. Most preferably, the spacer has a width of approximately 0.6 cm and a length of approximately 1 cm. The three-dimensional form spacer comprises a top portion and a bottom portion, preferably wherein the spacer defines at least one slit opening extending through the bottom portion and at least one slit opening extending through the top portion, each slit opening being sized and configured so that at least one retention strip and the attached filament can be threaded through the spacer. Also, preferably, the spacer further defines a window on each of two longitudinal sides, wherein the windows are sized and configured to provide access to the filament when the filament is disposed within the spacer and it is desired to sever the filament with a cutting device via an endoscope. The spacer can be comprised of any biocompatible material. Preferably, the spacer is comprised of a plastic material.

The step of inserting a spacer between the pierced stomach wall and the opposing stomach wall to maintain a prescribed distance between the inner surface of the pierced stomach wall and the inner surface of the opposing wall while in a passageway-restricting position may be done in any manner which maintains the prescribed distance between the opposing stomach walls. Preferably, the spacer is threaded down at least one retention strip and pushed down the retention strip and filament attached to the retention strip until the spacer touches the opposing stomach wall. Most preferably, the spacer is threaded down two retention strips and pushed down the retention strips and attached filaments until the spacer touches the opposing stomach wall.

Except as may be expressly otherwise indicated, the article “a” or “an” if and as used herein is not intended to limit, and should not be construed as limiting, the description or a claim to a single element to which the article refers. Rather, the article “a” or “an” if and as used herein is intended to cover one or more such elements, unless the text expressly indicates otherwise.

Each and every patent or other publication or published document referred to in any portion of this specification is incorporated in toto into this disclosure by reference, as if fully set forth herein.

This invention is susceptible to considerable variation and should not be considered limited to the particular exemplary embodiments described with detail hereinabove. Rather, the present invention is that which falls within the spirit and scope of the appended claims.

1. A method of performing a bariatric procedure comprising
piercing a wall of a stomach, the stomach defining a passageway,
disposing at least a portion of a restricting device through the pierced wall,
actuating the restricting device to draw the pierced wall and an opposing wall together in a passageway-restricting position, with or without piercing an exterior surface of the opposing wall, and
anchoring the restricting device to the stomach to maintain the pierced and opposing stomach walls in the passageway-restricting position.

2. A method according to claim 1 further comprising
inhibiting contact between an inner surface of the pierced stomach wall and an inner surface of the opposing stomach wall while the pierced and opposing stomach walls are drawn together and anchored in a passageway-restricting position.

3. A method according to claim 2 further comprising
inserting a spacer between the pierced stomach wall and the opposing stomach wall to maintain a prescribed distance between the inner surface of the pierced stomach wall and the inner surface of the opposing wall while in a passageway-restricting position.

4. The method according to claim 1 wherein the step of anchoring the restricting device to the stomach comprises attaching at least one wall restraining anchor to the opposing stomach wall.

5. The method according to claim 4 further comprising
advancing a retention device against an outside surface of the pierced wall of the stomach until the inner surface of the pierced wall of the stomach abuts at least a portion of a surface of the spacer, thereby bringing the pierced wall and the opposing wall of the stomach closer together in the passage-restricting position, while inhibiting adhesion between mucosa on the pierced and opposing walls of the stomach.

6. A medical device comprising: at least one anchor, an anchor deployment device, at least one filament, and a retention strip forming ridges on at least one surface of the retention strip, wherein the anchor is connected to the filament and the filament is connected to the retention strip, and wherein the anchor deployment device is sized and configured to be at least partially threaded over the anchor, the filament, and the retention strip so that, during use, the anchor can be deployed from the anchor deployment device into a stomach wall to a deployed position wherein the filament is attached to the anchor, the retention strip is attached to the filament, and the anchor deployment device is removable from a position surrounding the anchor, the attached filament, and the retention strip while the anchor, the filament, and the retention strip remain in the deployed position.

7. The medical device of claim 6, wherein the filament is comprised of a plastic material and has a diameter of approximately a 0-0 suture.

8. The medical device of claim 6, wherein the retention strip is comprised of a plastic material and wherein the ridges on the surface of the retention strip are sized and configured to operatively connect to a retention head in at least one opening of a retention device in order to allow irreversible ratcheting movement of the retention device down the retention strip.

9. The medical device of claim 7, wherein the anchor is comprised of a plastic material and forms one or more gripping teeth on at least one anchor surface.

10. The medical device of claim 7 further comprising a spacer having a three-dimensional form and comprising a top portion and a bottom portion, where in the spacer defines at least one slit opening extending through the bottom portion and at least one slit opening extending through the top portion, each slit opening being sized and configured so that at least one retention strip and the filament can be threaded through the spacer.

11. The medical device of claim 7, wherein the spacer further defines a window on each of two longitudinal sides, wherein the windows are sized and configured to provide access to the filament when the filament is disposed within the spacer and it is desired to sever the filament with a cutting device.

12. A medical device comprising: a rigid backing and a collagen plug connected thereto.

13. The medical device of claim 12, wherein the rigid backing and the collagen plug each define a respective opening, wherein the opening defined by the rigid backing is substantially aligned with the opening defined by the collagen plug.

14. The medical device of claim 13, wherein the opening defined by the rigid backing contains at least one retention head, wherein said retention head is sized and configured to catch at least one ridge on a surface of a retention strip when the retention strip is threaded through the opening defined by the rigid backing into a retention position, to thereby retain the retention strip in place.
